(19) United States
(12) Patent Application Publication
(10) Pub. No.: US 2010/0057197 A1
(43) Pub. Date: Mar. 4, 2010

(54) MEDICAL DEVICES HAVING INORGANIC COATINGS FOR THERAPEUTIC AGENT DELIVERY

(75) Inventors: Jan Weber, Maastricht (NL); Torsten Scheuermann, Munich (DE); Barry J. O'Brien, Galway (IE); Yixin Xu, Newton, MA (US); Bruce Forsyth, Hanover, MN (US)

Correspondence Address: MAYER & WILLIAMS PC, 251 NORTH AVENUE WEST, 2ND FLOOR, WESTFIELD, NJ, 07090, US

(73) Assignee: Boston Scientific Scimed, Inc., Maple Grove, MN (US)

(21) Appl. No.: 12/544,721

(22) Filed: Aug. 20, 2009

Related U.S. Application Data
(60) Provisional application No. 61/092,347, filed on Aug. 27, 2008.

(51) Int. Cl. A61F 2/82; A61N 1/05

(52) U.S. Cl. 623/1.42; 607/116; 623/1.46

(57) ABSTRACT

According to an aspect of the invention, medical devices are provided that comprise a substrate, at least one therapeutic agent disposed over or in the substrate, and at least one inorganic layer disposed over the therapeutic agent and the substrate, wherein the inorganic layer is either a porous inorganic layer or is a non-porous layer that becomes a porous inorganic layer in vivo. Other aspects of the invention comprise methods for forming medical devices.

### RELATED APPLICATIONS

This application claims priority from U.S. provisional application 61/092,347, filed Aug. 27, 2008, which is incorporated by reference herein in its entirety.

### TECHNICAL FIELD

This invention relates to medical devices, and more particularly, to medical devices having inorganic coatings that allow the release of underlying therapeutic agents.

### BACKGROUND OF THE INVENTION

The in-situ delivery of therapeutic agents within the body of a patient is common in the practice of modern medicine. In-situ delivery of therapeutic agents is often implemented using medical devices that may be temporarily or permanently placed at a target site within the body. These medical devices can be maintained, as required, at their target sites for short or prolonged periods of time, in order to deliver therapeutic agents to the target site.

For example, in recent years, drug eluting coronary stents, which are commercially available from Boston Scientific Corp. (TAXUS), Johnson & Johnson (CYPHER) and others, have been widely used for maintaining vessel patency after balloon angioplasty. These products are based on metallic expandable stents with biostable polymer coatings that release antirestenotic drugs at a controlled rate and total dose.

### SUMMARY OF THE INVENTION

According to an aspect of the invention, medical devices are provided that comprise a substrate, at least one therapeutic agent disposed over or in the substrate, and at least one inorganic layer disposed over the therapeutic agent and the substrate, wherein the inorganic layer is either a porous inorganic layer or is a non-porous layer that becomes a porous inorganic layer in vivo.

Other aspects of the invention comprise methods for forming medical devices.

An advantage of the present invention is that medical devices may be provided, in which the release of therapeutic agents is controlled.

Another advantage of the present invention is that therapeutic-agent releasing medical devices are provided, which have inorganic outer layers. Inorganic materials commonly have enhanced biocompatibility, including enhanced vascular biocompatibility.

Another advantage of the present invention is that medical devices with release-regulating inorganic layers may be provided, in which it is not necessary to pass therapeutic agent into or through the inorganic layers in order to load the medical devices with the therapeutic agent.

These and other embodiments and advantages of the present invention will become immediately apparent to those of ordinary skill in the art upon review of the Detailed Description and Claims to follow.

### BRIEF DESCRIPTION OF THE DRAWINGS

FIGS. 1-5 are schematic cross-sectional illustrations of medical devices in accordance with various embodiments of the invention.

FIG. 6A is a schematic cross-sectional illustration of a medical device in accordance with an embodiment of the invention. FIG. 6B is a schematic cross-section illustrating the medical device of FIG. 6A after being implanted or inserted into a subject for a period of time.

FIG. 7A is a schematic cross-sectional illustration of a medical device in accordance with an embodiment of the invention. FIG. 7B is a schematic cross-section illustrating the medical device of FIG. 7A after being implanted or inserted into a subject for a period of time.

FIG. 8 is a schematic illustration of an apparatus for forming medical devices in accordance with an embodiment of the invention.

FIG. 9A is a scanning electron micrograph (SEM) (5000×) of a substantially smooth coating. FIGS. 9B and 9C are SEMs of coatings that are suitable for use as rough underlying layers, in accordance with an embodiment of the invention.

FIG. 10A is a schematic cross-sectional illustration of a medical device prior to application of an inorganic surface layer, in accordance with an embodiment of the invention. FIG. 10B is a schematic cross-section illustrating the medical device of FIG. 10A, after application of an inorganic surface layer.

### DETAILED DESCRIPTION

According to an aspect of the invention, medical devices are provided that comprise a substrate, at least one therapeutic agent disposed over or in the substrate, and at least one inorganic layer disposed over the therapeutic agent and the substrate, wherein the inorganic layer is either a porous inorganic layer or is a non-porous layer that eventually becomes a porous inorganic layer in vivo (also referred to herein as a “pro-porous” inorganic layer).

In some embodiments, the inorganic layers are nanoporous inorganic layers. However, the present invention is not limited to nanoporous inorganic layers. Inorganic layers of any porosity may be employed.

Examples of medical devices benefiting from the present invention vary widely and include implantable or insertable medical devices, for example, stents (including coronary vascular stents, peripheral vascular stents, cerebral, urethral, ureteral, biliary, tracheal, gastrointestinal and esophageal stents), stent coverings, stent grafts, vascular grafts, abdominal aortic aneurysm (AAA) devices (e.g., AAA stents, AAA grafts), vascular access ports, dialysis ports, catheters (e.g., urological catheters or vascular catheters such as balloon catheters and various central venous catheters), guide wires, balloons, filters (e.g., vena cava filters and mesh filters for distil protection devices), embolization devices including cerebral aneurysm filler coils (including Guglielmi detachable coils and metal coils), septal defect closure devices, myocardial plugs, patches, electrical stimulation leads, including leads for pacemakers, leads for implantable cardioverter-defibrillators, leads for spinal cord stimulation systems, leads for deep brain stimulation systems, leads for peripheral nerve stimulation systems, leads for cochlear implants and leads for retinal implants, ventricular assist devices including left ventricular assist hearts and pumps, total artificial hearts, shunts, valves including heart valves and vascular valves, anastomosis clips and rings, tissue bulking devices, and tissue engineering scaffolds for cartilage, bone, skin and other in vivo tissue regeneration, sutures, suture anchors, tissue staples and ligating clips at surgical sites, cannulae, metal wire ligatures, urethral slings, hernia “meshes”, artificial ligaments, orthopedic prosthesis such as bone grafts, bone plates, fins and fusion devices, joint prostheses, orthopedic fixation devices such as interference screws in the ankle, knee, and hand areas, tacks for ligament attachment and meniscal repair, rods and pins for fracture fixation, screws and plates for craniomaxillofacial repair, dental implants, or other devices that are implanted or inserted into the body and from which therapeutic agent is released.

Thus, in some embodiments the devices of the invention may simply provide for therapeutic agent release, whereas in other embodiments, they are configured to provide a therapeutic function beyond controlled therapeutic agent release, for instance, providing mechanical, thermal, magnetic and/or electrical functions within the body, among other many possible functions.

The medical devices of the present invention include, for example, implantable and insertable medical devices that are used for systemic treatment, as well as those that are used for the localized treatment of any mammalian tissue or organ. Non-limiting examples are tumors; organs including the heart, coronary and peripheral vascular system (referred to overall as “the vasculature”), the urogenital system, including kidneys, bladder, urethra, ureters, prostate, vagina, uterus and ovaries, eyes, ears, spine, nervous system, lungs, trachea, esophagus, intestines, stomach, brain, liver and pancreas, skeletal muscle, smooth muscle, breast, dermal tissue, cartilage, tooth and bone.

As used herein, “treatment” refers to the prevention of a disease or condition, the reduction or elimination of symptoms associated with a disease or condition, or the substantial or complete elimination of a disease or condition. Subjects are vertebrate subjects, more typically mammalian subjects including human subjects, pets and livestock.

Substrate materials for the medical devices of the present invention may vary widely in composition and are not limited to any particular material. They can be selected from a range of biostable materials and biodisintegrable materials (i.e., materials that, upon placement in the body, are dissolved, degraded, resorbed, and/or otherwise removed from the device), including (a) organic materials (i.e., materials containing organic species, typically 50 wt % or more, for example, from 50 wt % to 75 wt % to 90 wt % to 95 wt % to 97.5 wt % to 99 wt % or more) such as polymeric materials (i.e., materials containing polymers, typically 50 wt % or more polymers, for example, from 50 wt % to 75 wt % to 90 wt % to 95 wt % to 97.5 wt % to 99 wt % or more) and biologics, (b) inorganic materials (i.e., materials containing inorganic species, typically 50 wt % or more, for example, from 50 wt % to 75 wt % to 90 wt % to 95 wt % to 97.5 wt % to 99 wt % or more), such as metallic inorganic materials (i.e., materials containing metals, typically 50 wt % or more, for example, from 50 wt % to 75 wt % to 90 wt % to 95 wt % to 97.5 wt % to 99 wt % or more) and non-metallic inorganic materials (i.e., materials containing non-metallic inorganic materials, typically 50 wt % or more, for example, from 50 wt % to 75 wt % to 90 wt % to 95 wt % to 97.5 wt % to 99 wt % or more) (e.g., including carbon, semiconductors, glasses and ceramics, which may contain various metal- and non-metal-oxides, various metal- and non-metal-nitrides, various metal- and non-metal-carbides, various metal- and non-metal-borides, various metal- and non-metal-phosphates, and various metal- and non-metal-sulfides, among others), and (c) hybrid materials (e.g., hybrid organic-inorganic materials, for instance, polymer/metallic inorganic and polymer/non-metallic inorganic hybrids).

Specific examples of inorganic non-metallic materials may be selected, for example, from materials containing one or more of the following: metal oxide ceramics, including aluminum oxides and transition metal oxides (e.g., oxides of titanium, zirconium, hafnium, tantalum, molybdenum, tungsten, rhenium, iron, niobium, and iridium); silicon; silicon-based ceramics, such as those containing silicon nitrides, silicon carbides and silicon oxides (sometimes referred to as glass ceramics); calcium phosphate ceramics (e.g., hydroxyapatite); carbon; and carbon-based, ceramic-like materials such as carbon nitrides.

Specific examples of metallic materials may be selected, for example, from metals such as gold, iron, niobium, platinum, palladium, iridium, osmium, rhodium, titanium, tantalum, tungsten, ruthenium, zinc, and magnesium, among others, and alloys such as those comprising iron and chromium (e.g., stainless steels, including platinum-enriched radiopaque stainless steel), niobium alloys, titanium alloys, alloys comprising nickel and titanium (e.g., Nitinol), alloys comprising cobalt and chromium, including alloys that comprise cobalt, chromium and iron (e.g., elgiloy alloys), alloys comprising nickel, cobalt and chromium (e.g., MP 35N), alloys comprising cobalt, chromium, tungsten and nickel (e.g., L605), alloys comprising nickel and chromium (e.g., inconel alloys), and biodisintegrable alloys including alloys of magnesium, zinc and/or iron (and their alloys with combinations of Ce, Ca, Al, Zr and Li), among others (e.g., alloys of magnesium including its alloys that comprises one or more of Fe, Ce, Al, Ca, Zn, Zr, La and Li, alloys of iron including its alloys that comprise one or more of Mg, Ce, Al, Ca, Zn, Zr, La and Li, alloys of zinc including its alloys that comprise one or more of Fe, Mg, Ce, Al, Ca, Zr, La and Li, etc.).

Specific examples of organic materials include polymers (which may be biostable or biodisintegrable) and other high and low molecular weight organic materials, and may be selected, for example, from suitable materials containing one or more of the following: polycarboxylic acid homopolymers and copolymers including polyacrylic acid, alkyl acrylate and alkyl methacrylate homopolymers and copolymers, including poly(methyl methacrylate-b-n-butyl acrylate-b-methyl methacrylate) and poly(styrene-b-n-butyl acrylate-b-styrene) triblock copolymers, polyamides including nylon 6,6, nylon 12, and polyether-block-polyamide copolymers (e.g., Pebax® resins), vinyl homopolymers and copolymers including polyvinyl alcohol, polyvinylpyrrolidone, polyvinyl halides such as polyvinyl chlorides and ethylene-vinyl acetate copolymers (EVA), vinyl aromatic homopolymers and copolymers such as polystyrene, styrene-maleic anhydride copolymers, vinyl aromatic-alkene copolymers including styrene-butadiene copolymers, styrene-ethylene-butylene copolymers (e.g., a poly(styrene-b-ethylene/butylene-b-styrene (SEBS) copolymer, available as Kraton® G series polymers), styrene-isoprene copolymers (e.g., poly(styrene-b-isoprene-b-styrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as poly(styrene-b-isobutylene-b-styrene) or SIBS, which is described, for instance, in U.S. Pat. No. 6,545,097 to Pinchuk et al.), ionomers, polyesters including polyethylene terephthalate and aliphatic polyesters such as homopolymers and copolymers of lactide (which includes d-,l- and meso-lactide) (e.g., poly(L-lactide) and poly(d,l-lactide), glycolide (glycolic acid), and epsilon-caprolactone, including poly(lactide-co-glycolides) such as poly(l-lactide-co-glycolide) and poly(d,l-lactide-co-glycolide), polycarbonates including trimethylene carbonate (and its alkyl derivatives), polyanhydrides, polyorthoesters, polyether homopolymers and copolymers including polyalkylene oxide polymers such as polyethylene oxide (PEO) and polyether ether ketones, polyolefin homopolymers and copolymers, including polyalkylenes such as polypropylene, polyethylene, polybutylenes (such as polybut-1-ene and polyisobutylene), polyolefin elastomers (e.g., santoprene) and ethylene propylene diene monomer (EPDM) rubbers, fluorinated homopolymers and copolymers, including polytetrafluoroethylene (PTFE), poly(tetrafluoroethylene-co-hexafluoropropene) (FEP), modified ethylene-tetrafluoroethylene copolymers (ETFE) and polyvinylidene fluoride (PVDF), silicone homopolymers and copolymers including polydimethylsiloxane, polyurethanes, biopolymers such as polypeptides, proteins, polysaccharides, fibrin, fibrinogen, collagen, elastin, chitosan, gelatin, starch, and glycosaminoglycans such as hyaluronic acid; as well as blends and further copolymers of the above.

The foregoing polymers may be provided in a number of configurations, which may be selected, for example, from cyclic, linear and branched configurations. Branched configurations include star-shaped configurations (e.g., configurations in which three or more chains emanate from a single branch point, such as a seed molecule), comb configurations (e.g., configurations having a main chain and a plurality of side chains), dendritic configurations (e.g., arborescent and hyperbranched polymers), networked (e.g., crosslinked) configurations, and so forth.

As indicated above, in one aspect of the invention, medical devices are provided that comprise, in addition to a substrate, at least one therapeutic agent disposed over or in the substrate, and at least one porous/pro-porous inorganic layer disposed over the therapeutic agent. In some embodiments, the therapeutic agent is provided within the substrate. In some embodiments, the therapeutic agent is provided in a distinct therapeutic-agent-containing layer (also referred to herein as a “therapeutic layer”) between the substrate and porous/pro-porous inorganic layer.

As used herein a “layer” of a given material is a region of that material whose thickness is small compared to both its length and width (e.g., its length and width are each at least four times as great as its thickness). Terms such as “film,” “layer” and “coating” may be used interchangeably herein. As used herein a layer need not be planar, for example, taking on the contours of an underlying substrate. A layer can be discontinuous, providing only partial coverage of an underlying structure (e.g., made up of a collection of two or more, sometimes many more, material regions).

For example, a layer may be provided over an underlying substrate in a desired pattern using suitable applicator (e.g., ink jet device, pen, brush, roller, etc.) or using a suitable masking technique. As a more specific example, in certain embodiments of the invention, a patterned therapeutic layer is provided over an underlying substrate. Because distinct surface regions of the substrate are not covered by the therapeutic layer in such embodiments, this may be advantageous, for example, in that direct contact (and bonding) is possible between the substrate and an overlying porous/pro-porous inorganic layer.

Therapeutic layer may contain, for example, from 1 wt % or less to 2 wt % to 5 wt % to 10 wt % to 25 wt % to 50 wt % to 75 wt % to 90 wt % to 95 wt % to 97.5 wt % to 99 wt % or more of a single therapeutic agent or of a mixture of therapeutic agents within the layer. Examples of additional materials other than therapeutic agent(s) which can be used to form therapeutic layers include materials that serve as reservoirs/binders/matrices for the therapeutic agent, including organic materials (e.g., polymeric materials, etc.), inorganic materials (e.g., metallic inorganic materials and non-metallic inorganic materials), and hybrid organic-inorganic materials, which may be selected, for example, from those listed above, among others. For example, the therapeutic layers may comprise one or more therapeutic agents blended with one or more additional materials, for instance, blended with organic materials, inorganic materials, or hybrids thereof. As another example, the therapeutic layers may comprise one or more therapeutic agents disposed within porous or nonporous reservoir layers formed from the additional materials, for instance, formed from organic materials, inorganic materials, or hybrids thereof. The therapeutic agent may be, for example, co-deposited with the additional material, or a layer of the additional materials be first deposited followed by introduction of the therapeutic agent to the additional material, among other possibilities.

Therapeutic layer thicknesses may vary widely, typically ranging from 10 nm to 100 nm to 1000 nm (1 μm) to 10000 nm (10 μm) or more in thickness.

In certain embodiments, the medical devices of the invention have sustained therapeutic agent release profiles. By “sustained release profile” is meant a release profile in which less than 25% of the total release from the medical article that occurs over the entire course of administration occurs over the first 1 day (or in some embodiments, over the first 2, 4, 8, 16, 32, 64, 128 or even more days) of administration. This means that more than 75% of the total release from the medical device will occur after the device has been administered for the same period (i.e., over the first 1, 2, 4, 8, 16, 32, 64, 128 or more days).

“Therapeutic agents,” “pharmaceutically active agents,” “pharmaceutically active materials,” “drugs,” “biologically active agents” and other related terms may be used interchangeably herein and include genetic therapeutic agents, non-genetic therapeutic agents and cells. A wide variety of therapeutic agents can be employed in conjunction with the present invention including those used for the treatment of a wide variety of diseases and conditions.

Exemplary therapeutic agents for use in connection with the present invention include: (a) anti-thrombotic agents such as heparin, heparin derivatives, urokinase, clopidogrel, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) smooth muscle relaxants such as alpha receptor antagonists (e.g., doxazosin, tamsulosin, terazosin, prazosin and alfuzosin), calcium channel blockers (e.g., verapimil, diltiazem, nifedipine, nicardipine, nimodipine and bepridil), beta receptor agonists (e.g., dobutamine and salmeterol), beta receptor antagonists (e.g., atenolol, metaprolol and butoxamine), angiotensin-II receptor antagonists (e.g., losartan, valsartan, irbesartan, candesartan, eprosartan and telmisartan), and antispasmodic/anticholinergic drugs (e.g., oxybutynin chloride, flavoxate, tolterodine, hyoscyamine sulfate, diclomine), (u) bARKct inhibitors, (v) phospholamban inhibitors, (w) Serca 2 gene/protein, (x) immune response modifiers including aminoquizolines, for instance, imidazoquinolines such as resiquimod and imiquimod, (y) human apolioproteins (e.g., AI, AII, AIII, AIV, AV, etc.), (z) selective estrogen receptor modulators (SERMs) such as raloxifene, lasofoxifene, arzoxifene, miproxifene, ospemifene, PKS 3741, MF 101 and SR 16234, (aa) PPAR agonists, including PPAR-alpha, gamma and delta agonists, such as rosiglitazone, pioglitazone, netoglitazone, fenofibrate, bexaotene, metaglidasen, rivoglitazone and tesaglitazar, (bb) prostaglandin E agonists, including PGE2 agonists, such as alprostadil or ONO 8815Ly, (cc) thrombin receptor activating peptide (TRAP), (dd) vasopeptidase inhibitors including benazepril, fosinopril, lisinopril, quinapril, ramipril, imidapril, delapril, moexipril and spirapril, (ee) thymosin beta 4, (ff) phospholipids including phosphorylcholine, phosphatidylinositol and phosphatidylcholine, (gg) VLA-4 antagonists and VCAM-1 antagonists.

Specific therapeutic agents include taxanes such as paclitaxel (including particulate forms thereof, for instance, protein-bound paclitaxel particles such as albumin-bound paclitaxel nanoparticles, e.g., ABRAXANE), sirolimus, everolimus, tacrolimus, zotarolimus, Epo D, dexamethasone, estradiol, halofuginone, cilostazole, geldanamycin, alagebrium chloride (ALT-711), ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, Serca 2 gene/protein, imiquimod, human apolioproteins (e.g., AI-AV), growth factors (e.g., VEGF-2) , as well derivatives of the forgoing, among others.

Numerous therapeutic agents, not necessarily exclusive of those listed above, have been identified as candidates for vascular treatment regimens, for example, as agents targeting restenosis (antirestenotics). Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists such as bosentan, sitaxsentan sodium, atrasentan, endonentan, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) Angiotensin Converting Enzyme (ACE) inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, atorvastatin, fluvastatin, simvastatin and cerivastatin, (u) fish oils and omega-3-fatty acids, (v) free-radical scavengers/antioxidants such as probucol, vitamins C and E, ebselen, trans-retinoic acid, SOD (orgotein) and SOD mimics, verteporfin, rostaporfin, AGI 1067, and M 40419, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) matrix metalloprotease (MMP) pathway inhibitors such as marimastat, ilomastat, metastat, batimastat, pentosan polysulfate, rebimastat, incyclinide, apratastat, PG 116800, RO 1130830 or ABT 518, (y) cell motility inhibitors such as cytochalasin B, (z) antiproliferative/antineoplastic agents including antimetabolites such as purine antagonists/analogs (e.g., 6-mercaptopurine and pro-drugs of 6-mercaptopurine such as azathioprine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), olimus family drugs (e.g., sirolimus, everolimus, tacrolimus, zotarolimus, etc.), cerivastatin, flavopiridol and suramin, (aa) matrix deposition/organization pathway inhibitors such as halofuginone or other quinazolinone derivatives, pirfenidone and tranilast, (bb) endothelialization facilitators such as VEGF and RGD peptide, (cc) blood rheology modulators such as pentoxifylline and (dd) glucose cross-link breakers such as alagebrium chloride (ALT-711).

Numerous additional therapeutic agents useful for the practice of the present invention are also disclosed in U.S. Pat. No. 5,733,925 to Kunz, the entire disclosure of which is incorporated by reference.

As previously indicated, in one aspect of the invention, medical devices are provided that comprise, in addition to a substrate and at least one therapeutic agent disposed over or in the substrate, at least one porous/pro-porous inorganic layer disposed over the therapeutic agent and the substrate.

Porous/pro-porous inorganic layers for use in the present invention may vary widely in composition and are not limited to any particular inorganic material. They can be selected from a wide range of biodisintegrable and biostable inorganic materials, such as suitable members of the inorganic materials listed above, including biostable metallic inorganic materials (e.g., titanium, iridium, tantalum, platinum, gold, niobium, molybdenum, rhenium, stainless steel, platinum-enriched radiopaque stainless steel, niobium alloys, titanium alloys, nitinol, etc.), biodisintegrable metallic inorganic materials (e.g., magnesium, iron, zinc, alloys of the same, etc.), and biostable and biodisintegrable non-metallic inorganic materials (e.g., titanium oxide, iridium oxide, aluminum oxide, iron oxide, silicon carbide, silicon nitride, titanium nitride, titanium oxy-nitride, calcium phosphate ceramics, etc.). Porous and pro-porous inorganic layers in accordance with the present invention may be, for example, fully biostable, fully biodisintegrable, or partially biostable and partially biodisintegrable.

The thickness of the porous/pro-porous inorganic layers for use in the present invention may vary widely, for example, ranging from 5 nm to 20 μm or more in layer thickness, among other values, for example, ranging from 5 nm to 10 nm to 100 nm to 1000 nm (1 μm) to 10000 nm (10 μm) or more in thickness.

In certain embodiments (e.g., porous/pro-porous inorganic layers formed using nanocluster PVD), the thicknesses of the porous/pro-porous inorganic layer will depend upon the size of the inorganic nanoparticles from which the inorganic layer is formed, in which case the layer thickness may range, for example, from 3 to 5 to 7 to 10 to 15 to 20 to 50 to 75 to 100 or more times the nanoparticle diameter. As used herein, a “nanoparticle” is a particle having a width that does not exceed 1 μm, for example, ranging from 2 nm or less to 4 nm to 8 nm to 10 nm to 15 nm to 20 nm to 25 nm to 35 nm to 50 nm to 100 nm to 150 nm to 250 nm to 500 nm to 1000 nm in width.

In some embodiments, the porous/pro-porous inorganic layers of devices of the present invention are either initially nanoporous or become nanoporous in vivo. In accordance with the International Union of Pure and Applied Chemistry (IUPAC), a “nanopore” is a pore having a width that does not exceed 50 nm (e.g., from 0.5 nm or less to 1 nm to 2.5 nm to 5 nm to 10 nm to 25 nm to 50 nm). As used herein, nanopores include “micropores,” which are pores having a width that does not exceed 2 nm, and “mesopores,” which are range from 2 to 50 nm in width. As used herein, “macropores” are larger than 50 nm in width and are thus not nanopores. In the present invention, “nanopores” may further embrace pores up to 1 μm in width, but only where this particular definition is explicitly invoked.

As used herein a “porous” layer is a layer that contains pores. A “nanoporous layer” is a layer that contains nanopores. Nanoporous layers may further comprise some pores that are not nanopores; for example, a nanoporous layer may further comprise macropores. Typically at least 90% by number of the pores within a nanoporous layer are nanopores.

Porous inorganic layers may be formed, for example, from biostable inorganic materials, a mixture of biostable and biodisintegrable inorganic materials, or biodisintegrable inorganic materials. Pro-porous inorganic layers may be formed, for example, from a mixture of biostable and biodisintegrable inorganic materials, or a mixture of biodisintegrable inorganic materials wherein one biodisintegrable inorganic material biodisintegrates faster than the other. For example, a layer may be formed with distinct biostable and biodisintegrable inorganic material phases, wherein the phase morphology is such that a porous layer is formed upon removal of the biodisintegrable phase in vivo. Porous and pro-porous inorganic layers may be formed, for example, using any suitable technique, including deposition techniques such as those described below.

In some embodiments, a biodisintegrable material (e.g., a biodisintegrable organic material, inorganic material, or organic-inorganic hybrid) is placed beneath the porous/pro-porous inorganic layer and over the therapeutic agent (i.e., between the therapeutic layer and the porous/pro-porous inorganic layer). In these embodiments the rate of release of the therapeutic agent may be dictated by the porous/pro-porous inorganic layer, by the biodisintegrable material, or both. Moreover, the porous/pro-porous inorganic layer may act as a barrier that prevents fragments of the biodisintegrable material from being released from the device.

In various embodiments of the invention, the porous/pro-porous inorganic layers are rough layers. Rough inorganic layers may, for example, be resistant to damage to the inorganic layer due to cracking, which may otherwise occur with a smoother layer. Without wishing to be bound by theory, this behavior may be explained in the following fashion. In the instance of a rough layer of relative constant thickness disposed over an underlying rough region versus a smooth layer of relatively constant thickness disposed over an underlying smooth region, the latter layer is believed to be more prone to cracking due to increased tensile stress (leading to cohesive failure) and interfacial stress. Moreover, cracking may propagate through a smooth layer as a result of poor substrate adhesion. Furthermore, rough layers may comprise numerous islands of inorganic material of thicker section (e.g., laterally spaced quasi-islands of relatively thick inorganic dots) connected regions of substantially thinner section. The thinner a material region, the lower the tensile/compression stresses on opposing surfaces of the material region upon bending. Conversely, the thicker the material region, the higher the tensile/compression stresses on opposing surfaces upon bending. (This is why a thin glass fiber is quite flexible, while a rod of the same material will break when flexed.) Consequently, when a layer with thicker and thinner regions is bent, the bending stresses tend to be absorbed by the thinner regions.

A “rough” region is determined by surface topography measurements (e.g. AFM) to be a region where the Sa value (i.e., the average roughness evaluated over the surface of the material, which can be mathematically expressed as follows: Sₐ=∫∫ₐ|Z(x, y)|dxdy) is greater than 50 nanometers (a typical electro-polished surface has a roughness value Sa on the order of 20-40 nanometers), typically greater than 100 nanometers, more typically greater than 300 nm. In this regard, with increasing surface roughness, one switches from shiny/glossy to dull as one passes about 300 nm in average roughness. A “rough” region may also be determined to be one whose surface has a Summit Density (S_{ds}), which is the number of peaks per unit area of the surface, of at least 20 1/μm². For further information on roughness testing see, e.g., ASME B46.1.

In certain embodiments, the porous/pro-porous inorganic layer is placed over only certain surfaces of the substrate. For instance, porous/pro-porous inorganic layers may be provided only on the outer/abluminal surface of tubular medical devices such as stents or only on the inner/luminal surfaces of such devices.

Where a porous/pro-porous inorganic layer is sufficiently thin, roughness may be imparted to the inorganic layer, for example, by means of a rough underlying material.

As discussed further below, where line-of-sight processes such as PVD-based processes (e.g., pulsed laser deposition, nanocluster PVD, etc.) are employed in the formation of a porous/pro-porous inorganic layer over a rough underlying material, the roughness of the underlying material can lead to incomplete coverage of the underlying material and the creation of a porous inorganic layer.

In some embodiments, the rough underlying material corresponds to a rough substrate material. Examples of such materials include substrates that are rough as formed (e.g., cast from a mold having a rough surface, etc.) and substrates that are roughened by a suitable roughening process after their formation. For example, a plasma immersion ion implantation (PIII) process may be used to roughen the surface of a metallic substrate, among many other processes, including, for example, chemical etching.

In some embodiments, the rough underlying material corresponds to a rough layer of material that is disposed over the substrate. Various processes are known for producing rough organic, inorganic and organic-inorganic hybrid layers over underlying substrates. Such rough layers may be biodisintegrable, biostable, or partially biodisintegrable and partially biostable.

In certain embodiments of the invention, an electrostatic spray (“electrospray”) coating process is employed to create a rough layer of material on a substrate. Information on electrospray processing may be found, for example, in Pub. No. US 2007/0048452 to Feng et al.

An electrospray coating method is described in the following paragraphs, whereby the final coating morphology can be controlled, for example, producing porous surface regions of partially fused polymeric particles (e.g., bridged/interconnected fibers, bridged/interconnected particles of low aspect ratio, etc.), smooth surface regions, or a combination or both as a function of layer depth. Typical particle sizes range from 15 to 2000 nm in diameter, among other possibilities, for example, from 15 to 20 to 50 to 100 to 200 to 500 to 1000 to 2000 nm in diameter. Partially fused particles can be produced very uniformly (monodisperse), can have spherical or non-spherical shapes and/or can be endowed with multiple structural properties (e.g., solid, encapsulated, hollow, dimpled, etc.). Thus, in some embodiments, a majority of the partially fused polymer particles have a low aspect ratio, for example, having an aspect ratio of two or less (see, e.g., FIG. 9B below). In some embodiments, a coating is applied to a substrate, such that the initial coating parameters optimize wetting or adherence to the substrate and subsequent coating parameters optimize porosity. In some embodiments, the morphology of the coating may be modified to mimic the morphology of natural tissue, thereby encouraging cell growth (e.g., endothelial cell growth) on the device.

In a specific example, SIBS and optionally a therapeutic agent such as paclitaxel (e.g., solids content consisting of 100 wt % SIBS or of 8.8 wt % paclitaxel and 91.2 wt % SIBS), may be deposited via electrospray processing from various solutions (e.g., those with overall solids concentration ranging from 1 wt % to 2.5 wt % to 5 wt %), for example, tetrahydrofuran (THF) rich solutions such as those employing THF alone as a solvent species (100 wt % THF as solvent species), THF blended with methanol (MeOH) (e.g., 85 wt % THF and 15 wt % MeOH as solvent species), THF blended with propylene carbonate (PC) (e.g., 97 wt % THF and 3 wt % PC as solvent species) and THF blended with methyl ethyl ketone (MEK) (e.g., 70 wt % THF and 30 wt % MEK as solvent species). Where a therapeutic agent is included in the coating, release profiles can be varied by varying the solvent composition. (Release may be further modulated by adding toluene to the preceding toluene rich solutions.) For example, by varying solution composition (solids content and solvent species), cumulative release of paclitaxel from SIBS after 10 days can be modulated between 10% and 90%, with some coatings demonstrating a substantially linear release profiles between 1 and 10 days.

Charging methods for electrospray processes include electrostatic induction charging and corona charging, such as with flow limited field ejection electrospray (FFESS), as is well known in the electrospray art. Process variables include applied voltage, solution flow rate, solution conductivity, target distance, gas temperature and capillary size. Varying levels of porosity within the coating can be affected, for instance, by varying the drying rate of the microdroplets that are formed in the electrospray process. For example, increasing the carrier gas temperature can assist in solvent drying, increasing the drying rate and producing more porous coatings, decreasing the capillary to target distance reduces solvent evaporation (producing a smoother coating), and increasing the capillary to target distance increases solvent evaporation (producing a more porous coating) but also requires an increase in applied voltage to maintain the same electric field strength for good cone-jet performance. Also, nitrogen gas with a modest amount of heat can increase the overall thermal energy of the sprayed solution, leading to enhanced evaporation. In a specific example, FIGS. 9A-9C represent scanning electron micrographs (SEMs) (5000×) of coatings formed on a flat metal (stainless steel 316L) coupon from a solution containing 85 wt % THF, 14 wt % MeOH and 1 wt % SIBS, using three differing sets of electrospray process variables. In this regard, processes that generate sub-micron droplets can generally be modulated via solution flow rate, applied potential/voltage, capillary nozzle-to-substrate distance and drying conditions (e.g., coflow gas and temperature). In combination with formulation parameters (e.g., solids, solvent blends, conductivity, etc.), various unique coating structures can be constructed. FIG. 9A is a substantially smooth morphology (an intentional scratch is seen at the right-hand side of the figure), whereas FIG. 9B is based on an interconnected particle (e.g., partially fused particles) morphology. The morphology of FIG. 9C is a example of a fused fibroid wherein a network of long aspect ratio particles are designed to coalesce and dry into a pattern with both high void regions and a high degree of solid interconnectivity (e.g., an open-porous foam).

In some embodiments, only a portion of a medical device is coated via electrospray processing. For example, a stent may be selectively coated on its outer/abluminal surface using insulative mandrels (thereby masking the inner/luminal surface) or using biased mandrels (to apply a repulsive electrical field).

In certain embodiments, a rough a porous/pro-porous inorganic layer is produced by deposition over a rough polymeric layer like that described above. The therapeutic agent may be provided, for example, within the rough polymeric layer, within a separate layer that is disposed in the pores of/over the rough polymeric layer, and so forth.

In certain embodiments, a rough a porous/pro-porous inorganic layer is produced by deposition over a rough inorganic layer. For example, a rough inorganic layer may be formed by first forming a rough polymeric layer like that described above. Then, a rough sol-gel derived ceramic layer is formed by first depositing a metallic or semi-metallic oxide gel on the rough polymeric layer, followed by calcining at high temperature, which strengthens the gel and bums off the polymeric component. A rough a porous/pro-porous inorganic layer is produced by deposition over the rough sol-gel derived layer. The therapeutic agent may be provided, for example, within the rough sol-gel derived layer, within a separate layer that is disposed in the pores of/over the rough sol-gel derived layer, and so forth.

By way of background, in a typical sol-gel process, precursor materials, typically selected from inorganic metallic and semi-metallic salts, metallic and semi-metallic complexes/chelates, metallic and semi-metallic hydroxides, and organometallic and organo-semi-metallic compounds such as metal alkoxides and alkoxysilanes, are subjected to hydrolysis and condensation (also referred to sometimes as “polymerization”) reactions, thereby forming a “sol” (i.e., a suspension of solid particles within a liquid). For example, an alkoxide of choice (e.g., a methoxide, ethoxide, isopropoxide, tert-butoxide, etc.) of a semi-metal or metal of choice (e.g., silicon, germanium, aluminum, zirconium, titanium, iron, hafnium, tantalum, molybdenum, tungsten, rhenium, iridium, barium, etc.) may be dissolved in a suitable solvent, for example, in one or more alcohols. Subsequently, water or another aqueous solution such as an acidic or basic aqueous solution (which aqueous solution can further contain organic solvent species such as alcohols) is added, causing hydrolysis and condensation to occur. Further processing of the sol enables solid materials to be made. For instance, “wet gel” coatings can be produced on an underlying structure by introducing a sol to the structure, for example, by dipping, spray coating, coating with an applicator (e.g., by roller, brush or pen), ink-jet printing, screen printing, and so forth. The wet gel is then dried. Drying at ambient temperature and ambient pressure leads to what is commonly referred to as a “xerogel.” Other drying possibilities are available including supercritical drying (producing an “aerogel”), freeze drying (producing a “cryogel”), elevated temperature drying (e.g., in an oven), vacuum drying (e.g., at ambient or elevated temperatures), and so forth. Further information concerning sol-gel materials can be found, for example, in Viitala R. et al., “Surface properties of in vitro bioactive and non-bioactive sol-gel derived materials,” Biomaterials, August 2002; 23(15):3073-86.

As previously indicated, therapeutic layers can be incorporated into the structures of the invention in various ways.

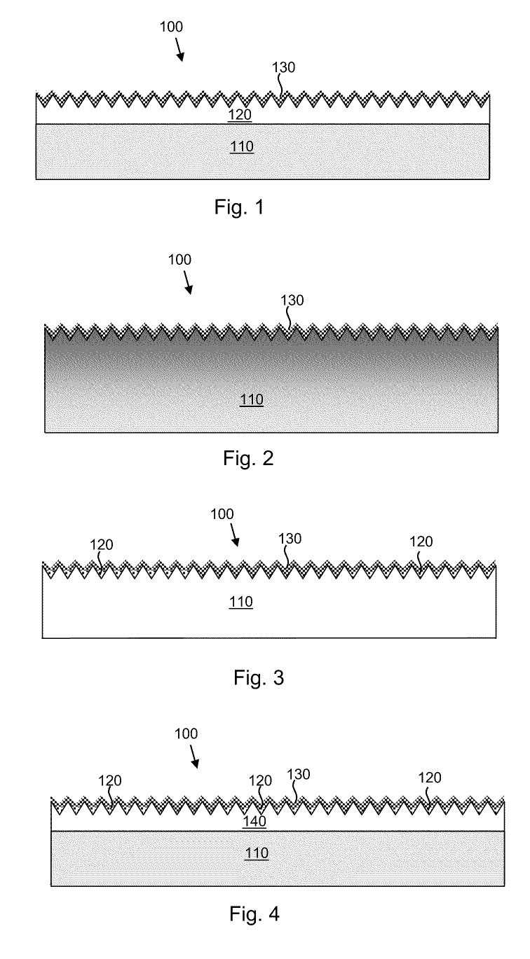

For example, at least one therapeutic agent may be included in a deposition material that is used to form a rough layer, thereby incorporating the therapeutic agent in the rough layer at the time of formation. A medical device of this type is schematically illustrated in FIG. 1, which shows a medical device 100 that comprises a substrate 110 (e.g., a stainless steel substrate, etc.), a rough therapeutic layer 120 disposed over the substrate 110, and a porous/pro-porous inorganic layer 130 (e.g., a PVD iridium layer, etc.) disposed over the therapeutic layer 120 and the substrate 110. The rough therapeutic layer 120 consists of at least one therapeutic agent or comprises at least one therapeutic agent and at least one additional material (e.g., a material that serves as a reservoir/binder/matrix for the therapeutic agent). One specific example of a rough therapeutic layer is an electrosprayed SIBS/paclitaxel layer such as that described above.

Examples of such additional materials include biostable and biodisintegrable organic and inorganic materials, which may be selected from those described above, among others. Such additional materials may thus be biodisintegrable, biostable, or partially biodisintegrable and partially biostable.

As another example, a composition containing at least one therapeutic agent (e.g., a powder, a solution, a liquid suspension, a melt, etc.) and any optional additional materials (e.g., materials that serve as reservoirs/binders/matrices for the therapeutic agent, solvent species, etc.) may be applied to a rough substrate or to a rough layer on a substrate.

In some embodiments, depending on the nature of the rough substrate or rough layer and on the nature of the applied composition, the therapeutic agent may be incorporated into the rough layer or rough substrate (or at least the surface portion of the rough layer or rough substrate). For example, the applied composition may be introduced into pores that are associated with the rough substrate or rough layer. As another example, the applied composition may be a solution, in which the therapeutic agent is dissolved in a solvent system that is also a swelling agent for the material forming the rough substrate or rough layer. This solution may be applied to the rough substrate or rough layer such that the rough substrate or rough layer is swollen by the solution, thereby uptaking the therapeutic agent contained therein.

A structure of this type is shown is shown schematically in FIG. 2, which shows a medical device 100 that comprises a rough substrate 110 and a porous/pro-porous inorganic layer 130 disposed over the substrate 110. In the structure of FIG. 2, the therapeutic agent, which has been introduced into to upper portion of the rough substrate 110, is depicted by the more darkly shaded portion of the rough substrate 110.

In other embodiments, the applied composition yields a distinct therapeutic layer on the surface of the rough substrate or rough layer. For example, the therapeutic layer may consist of a single therapeutic agent (or a mixture of therapeutic agents) in substantially pure form (i.e., without an additional material that is not a therapeutic agent). As another example, the therapeutic layer may include at least one therapeutic agent in combination with at least one additional material (e.g., a material that serves as a reservoir/binder/matrix for the therapeutic agent, such as those described above).

One example of such a medical device is schematically illustrated in FIG. 3, which shows a medical device 100 in accordance with an embodiment of the invention. The medical device shown comprises a rough substrate 110, a therapeutic layer 120 disposed over the rough substrate 110 (specifically two therapeutic-agent-containing regions, each constituting a portion of a patterned therapeutic layer 120, are shown), and a porous/pro-porous inorganic layer 130 over the therapeutic layer 120 and the substrate 110.

Another example of such a medical device is schematically illustrated in FIG. 4, which shows a medical device 100 in accordance with an embodiment of the invention. The medical device 100 shown comprises a substrate 110, rough layer 140 disposed over the substrate, a therapeutic layer 120 (three therapeutic-agent-containing regions, each constituting a portion of the therapeutic layer 120, are shown) disposed over the rough layer 140, and a porous/pro-porous inorganic layer 130 disposed over the therapeutic layer 120, rough layer 140 and the substrate 110.

As indicated above, additional materials for use in therapeutic layers may vary widely and include organic and inorganic materials. In certain embodiments, the additional materials may be selected from sol-gel derived metallic and non-metallic oxides. For example, at least one therapeutic agent may be, for example, combined with a sol or sol precursor (e.g., metal or semi-metal alkoxide solution), which is subsequently used to form a gel layer on a rough layer or rough substrate. Alternatively, at least one therapeutic agent (e.g., in the form of a solution or a suspension) may be introduced to a previously formed gel, in which case the gel may be subjected to elevated temperatures (e.g., in order to calcinate and strengthen the gel) prior to contact with the therapeutic agent. Such temperatures could otherwise destroy the therapeutic agent.

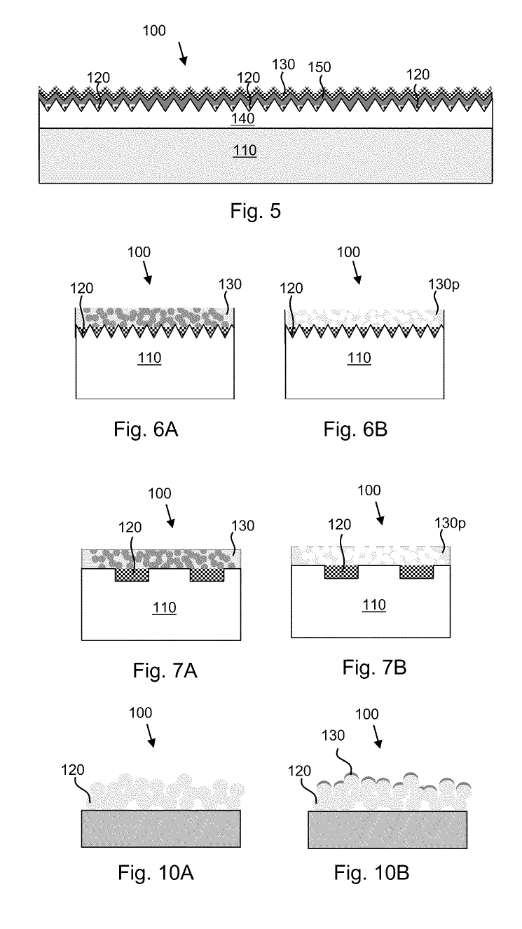

As previously indicated, a supplemental layer of material, for example, a fully or partially biodegradable organic or inorganic material layer or a porous organic or inorganic material layer, may be provided between the therapeutic layer and the porous/pro-porous inorganic layer, for example, in order to slow the release of the therapeutic agent. An example of such a structure is shown in FIG. 5, which is similar to FIG. 4, except that a supplemental layer of material 150 is disposed beneath the porous/pro-porous inorganic layer 130.

As noted above, in some embodiments, the porous/pro-porous inorganic layers described herein are advantageous in that they can act to prevent fragments of biodegradable materials disposed under the same from escaping the device. Moreover, in some embodiments, the porous/pro-porous inorganic layers described herein are advantageous in that they can shield underlying materials (e.g., a substrate material, a material used to form the rough layer, an additional material associated with the therapeutic layer, etc.) from direct contact with a subject into which the device is implanted. For example, an underlying material within the device may be one that results in thrombosis upon direct contact with the bloodstream, but which does not cause such an effect in the presence of an overlying porous inorganic layer.

In some aspects of the invention, the overlying inorganic layer is a smooth pro-porous layer. By “smooth” is meant a region whose surface roughness lies below the Sa values set forth above which define a “rough” surface. In many embodiments, a smooth surface will be glossy, in which case the surface structure has lateral discontinuities below the optical wavelength (e.g., an Sa value below about 300 nm).

A smooth surface layer may be desirable under a variety of circumstances. As one example, it may be desirable to provide a smooth layer on a stent, particularly the luminal surface of a stent, so as to avoid the possibility of balloon damage that may be attendant to the presence of a rough surface layer. Moreover, because the pro-porous layer is not initially porous, it may act to protect the underlying therapeutic agent from external conditions (e.g., exposure to ethylene oxide during device sterilization, etc.) in certain embodiments. In some embodiments, a medical device having a porous layer is subjected to a sterilization cycle prior to loading the porous layer with a therapeutic agent, after which the therapeutic-agent-loaded layer is closed off by an additional layer (e.g., a biodisintegrable layer or a pro-porous layer, which may be further subjected to an additional sterilization step).

In certain embodiments, the pro-porous layer has a configuration that allows the electropolishing of the layer to achieve a smooth surface. For example, the outermost surface of a porous or pro-porous layer may be covered in a biodegradable metal (e.g., magnesium or a magnesium alloy), which is then electropolished. The magnesium surface then biodisintegrates in vivo, allowing release the agent. Where immediate release of therapeutic agent is required, certain portions of the biodegradable metal may be etched away (while protecting/masking the smooth surfaces), followed by therapeutic agent loading in certain embodiments.

Pro-porous layers in accordance with the invention may comprise, for example, both biodisintegrable and biostable phases. Upon placement in vivo, the device ultimately develops pores, allowing the therapeutic agent to be released. In the case of a stent or another vascular medical device, the development of porosity may promote endothelial cell growth. In this regard, submicron topography, including pores, fibers, and elevations in the sub-100 nm range, has been observed for the basement membrane of the aortic valve endothelium as well as for other basement membrane materials. See R. G. Flemming et al., Biomaterials 20 (1999) 573-588, S. Brody et al., Tissue Eng. February 2006; 12(2): 413-421, and S. L. Goodman et al., Biomaterials 1996; 17: 2087-95. Goodman et al. employed polymer casting to replicate the topographical features of the subendothelial extracellular matrix surface of denuded and distended blood vessels, and they found that endothelial cells grown on such materials spread faster and appeared more like cells in their native arteries than did cells grown on untextured surfaces.

An example of a device having a smooth pro-porous layer is schematically illustrated in FIG. 6A. A medical device 100 is shown, which comprises a rough substrate 110 (e.g., a stainless steel substrate roughened by a PIII process, etc.), a therapeutic layer 120 (e.g., a layer of pure therapeutic agent such as a layer of paclitaxel or everolimus, etc.) disposed within the crevasses of the rough substrate 110, and a smooth pro-porous inorganic layer 130 (e.g., a layer comprising a biostable metallic phase such as an iridium phase, shown in light grey, and a biodisintegrable metallic phase such as a magnesium phase, shown in dark grey) disposed over the therapeutic layer 120 and rough substrate 110. As discussed below, such a layer 130 may be formed via PVD using a mixed composition target. Upon insertion of the device 100 into a subject, at least a portion of the biodisintegrable metallic phase is removed, leaving behind a porous layer 130p as shown in FIG. 6B, allowing the release of the therapeutic agent from the device.

As indicated above, in some embodiments, an additional material may be admixed with the therapeutic agent in the therapeutic layer and/or a supplemental layer may be disposed over the therapeutic layer. In these embodiments release profile of the therapeutic agent may be dictated by the pro-porous inorganic layer and by the additional material and/or supplemental layer. Moreover, the pro-porous inorganic layer can act as a barrier that prevents fragments of any underlying biodisintegrable materials from being released from the device.

In certain embodiments, the therapeutic agent is provided within surface depressions in the substrate. For example, a medical device 100 is illustrated schematically in FIG. 7A, which comprises a substrate 110 and a therapeutic layer 120 disposed within a series of depressions within the substrate 110. A smooth pro-porous inorganic layer 130 (e.g., like that described above in connection with FIG. 6A) is disposed over the therapeutic layer 120 and substrate 110. As with the device of FIG. 6A, upon insertion of the device into a subject, a porous layer 130p is formed in vivo as shown in FIG. 7B, allowing the release of the therapeutic agent from the device.

Examples of depressions include trenches, blind holes and pores, among others. Depressions may be created in a great variety of shapes and sizes. Multiple depressions can be provided in a near infinite variety of arrays. Examples of blind holes include those whose lateral dimensions at the surface are circular, polygonal (e.g., triangular, quadrilateral, penta-lateral, etc.), as well as blind holes of various other regular and irregular shapes and sizes. Trenches include simple linear trenches, wavy trenches, trenches formed from linear segments whose direction undergoes an angular change (e.g., zigzag trenches), and linear trench networks intersecting various angles, as well as other regular and irregular trench configurations. The depressions can be of any suitable size. For example, the medical devices of the invention typically contain depressions whose smallest lateral dimension (e.g., the width) is less than 10 mm (10000 μm), for example, ranging from 10000 μm to 1000 μm to 100 μm to 10 μm to 1 μm to 100 nm or less.

Examples of techniques for forming depressions (e.g., pores, blind holes, trenches, etc.) include methods in which a material contains depressions as-formed. These include molding techniques in which a mold may be provided with various protrusions, which after casting the substrate of interest, create depressions in the material. These techniques further include techniques, such as foam-based techniques, whereby a porous material is formed. Porous materials may also be formed by removing one component from a multi-component material using a suitable process (e.g., dissolution, etching, etc.). Examples of techniques for forming depressions further include direct removal techniques as well as mask-based removal techniques, in which masking is used to protect material that is not to be removed. Direct removal techniques include those in which material is removed through contact with solid tools (e.g., microdrilling, micromachining, etc.) and those that remove material without the need for solid tools (e.g., those based on directed energetic beams such as laser, electron, and ion beams). Mask-based techniques include those in which the masking material contacts the material to be machined (e.g., where masks are formed using known lithographic techniques) and techniques in which the masking material does not contact the material to be machined, but which is provided between a directed source of excavating energy and the material to be machined (e.g., opaque masks having apertures formed therein, as well as semi-transparent masks such as gray-scale masks which provide variable beam intensity and thus variable machining rates). Material is removed in regions not protected by the above masks using any of a range of processes including physical processes (e.g., thermal sublimation and/or vaporization of the material that is removed), chemical processes (e.g., chemical breakdown and/or reaction of the material that is removed), or a combination of both. Specific examples of removal processes include wet and dry (plasma) etching techniques, and ablation techniques based on directed energetic beams such as electron, ion and laser beams. In still other embodiments, depressions may be formed by selective growth of a material on a substrate surface, for example, on a patterned surface or on a masked surface.

Various methods for forming porous and pro-porous inorganic layers will now be described. For example, in some embodiments, the layers may be formed via vapor deposition methods, including physical vapor deposition (PVD) techniques. PVD processes are processes in which a source of material, typically a solid material, is vaporized, and transported to a structure upon which a film (i.e., a layer) of the material is formed. In the present invention, the solid material may be, for example, a biodisintegrable inorganic material, biostable inorganic material, or a combination of biodisintegrable and biostable inorganic materials.

PVD processes are generally used to deposit films with thicknesses in the range of a few nanometers to thousands of nanometers, although greater thicknesses are possible. PVD is typically carried out under vacuum (i.e., at pressures that are less than ambient atmospheric pressure). In many embodiments, the pressure associated with PVD techniques is sufficiently low such that little or no collisions occur between the vaporized source material and ambient gas molecules while traveling to the substrate. Hence, the trajectory of the vapor is generally a straight (line-of-sight) trajectory.

In certain embodiments, the PVD processing parameters are selected to form a porous layer. For example, as noted above, where line-of-sight processes such as PVD-based processes are employed in the formation of an inorganic layer over a rough underlying material, the roughness of the underlying material can lead to incomplete coverage of the underlying material and the creation of a porous inorganic layer. This is shown schematically in FIGS. 10A-10B. FIG. 10A is an illustration of a medical device 100 comprising a substrate 110 and a rough therapeutic layer 120, for example, a layer of partially fused polymeric particles that act as a matrix for a therapeutic agent (e.g., electrosprayed SIBS/paclitaxel, etc.). As shown in FIG. 10B, PVD-based deposition of an inorganic layer 130 (e.g., an iridium layer, etc.) results in substantial, but incomplete, coverage of the therapeutic layer 120 such that the inorganic layer 130 is porous. (On the other hand, if deposition is continued long enough, a smooth, thick non-porous inorganic layer will ultimately be formed.)

In other embodiments, the PVD processing parameters are selected to form a pro-porous layer. For example, a biostable metal and a biodisintegrable metal may be co-deposited such that a layer is formed with distinct biostable and biodisintegrable metal phases, whose phase morphology is such that a porous layer is formed upon biodisintegration and removal of the biodisintegrable metal phase in vivo.

Some specific PVD methods that are used to form porous/pro-porous layers in accordance with the present invention include evaporation, sublimation, sputter deposition and laser ablation deposition. For instance, in some embodiments, at least one source material is evaporated or sublimed, and the resultant vapor travels from the source to a substrate, resulting in a deposited layer on the substrate. Examples of sources for these processes include resistively heated sources, heated boats and heated crucibles, among others. Sputter deposition is another PVD process, in which surface atoms or molecules are physically ejected from a surface by bombarding the surface (commonly known as a “target”) with high-energy ions. Ions for sputtering can be produced using a variety of techniques, including arc formation (e.g., diode sputtering), transverse magnetic fields (e.g., magnetron sputtering), and extraction from glow discharges (e.g., ion beam sputtering), among others.

Pulsed laser deposition (PLD) is yet another PVD process, which is similar to sputter deposition, except that vaporized material is produced by directing laser radiation (e.g., pulsed laser radiation), rather than high-energy ions, onto the target material. As advantage of the PLD process is that films can be deposited upon substrates at or near room. Consequently, films can be formed over temperature-sensitive materials, for example, organic materials such as polymers and therapeutic agents.

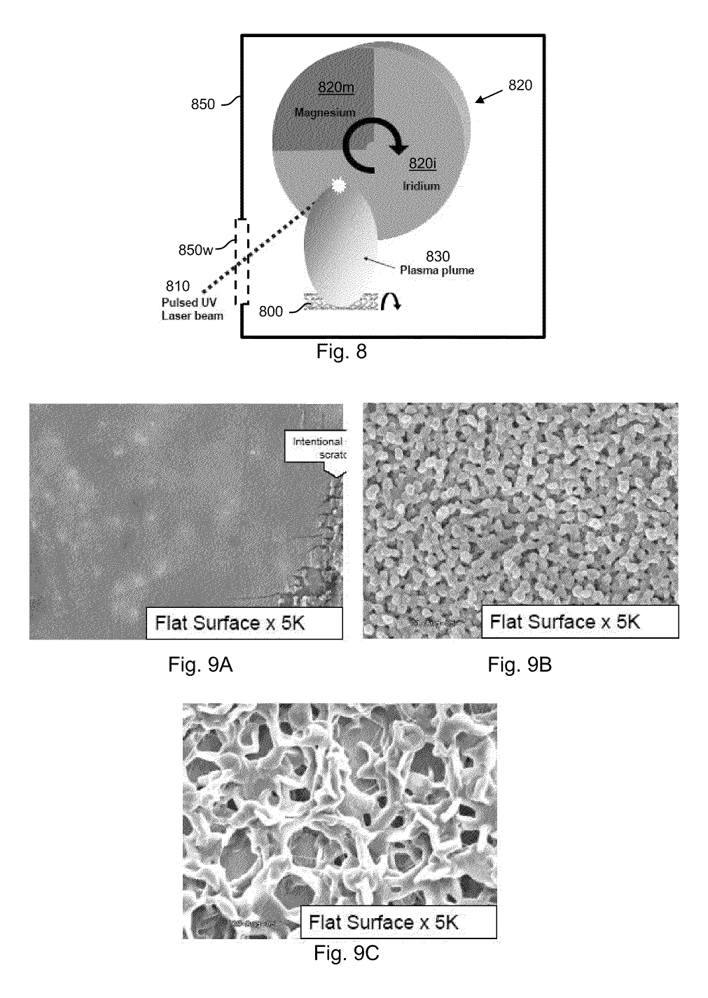

In a typical PLD process, and with reference to the schematic illustration of FIG. 8, a laser pulse 810 is directed into a vacuum chamber 850 through a window 850w and impinges onto a target material 820 to be deposited. The laser pulse 810 vaporizes the target material 820, forming a plume 830 that contains various species (e.g., neutral, ionic, molecular, etc.). These species travel toward a substrate, in this case, a rotating stent 800, and are deposited on the stent 800 in the form of a thin film. (If desired, the stent 800 may also be reciprocated longitudinally to improve coverage.) Targets include targets formed from a single material (e.g., a single metal or metal oxide) and targets formed from a multiple materials (e.g., multiple metals or multiple metal oxides). For example, the target 820 shown in FIG. 8 is a rotating target that comprises two materials, magnesium 820m and iridium 820i. Consequently, the film deposited on the rotating stent 800 contains magnesium and iridium. When the magnesium is removed upon implantation in a subject, a porous iridium layer is formed, as previously described.

As an alternative to an apparatus like that of FIG. 8, a dual beam set-up for simultaneous Mg/Ir deposition may be used, in which a first beam strikes an Mg target or an Mg region of a Mg—Ir composite target and a second beam strikes an Ir target or an Ir region of a composite target. This leads to a simultaneous deposition of both materials with layer thickness and composition depending on laser intensity per spot, distance to substrate, material type, and so forth.

As noted above, in certain embodiments, porous and pro-porous inorganic layers are formed from inorganic particles, which may be, for example, biodisintegrable inorganic particles, biostable inorganic particles, or a combination of biodisintegrable and biostable inorganic particles. In some embodiments at least some of the particles have the same composition as the underlying medical device substrate. Specific examples include iridium, tantalum, titanium, cobalt, iron, zinc, gold, alloys containing two or more of the same, stainless steel and nitinol.

Methods of forming porous/pro-porous inorganic layers in accordance with the present invention include those wherein inorganic nanoparticles are created, accelerated and directed onto upper surfaces of structures, thereby forming inorganic layers over the structures. For example, in some embodiments, the nanoparticles are charged nanoparticles, which are accelerated onto a structure surface by subjecting them to an electric field. The trajectory of the nanoparticles may be further influenced through the use of a secondary electric field or a magnetic field, where desired. In some embodiments, the nanoparticles are magnetic or ferromagnetic nanoparticles, which are accelerated onto a structure surface by subjecting them to a suitable magnetic field. The trajectory of the nanoparticles may be further influenced through the use of a secondary magnetic field, where desired.

Without wishing to be bound by theory, when nanoparticles are accelerated towards a surface (e.g., in a magnetic field, electrical field, etc.), melting can be induced upon landing by imparting them with sufficient kinetic energy. As seen from the above, there are various ways to accelerate nanoparticles toward a structure. For example, in embodiments where charged nanoparticles are accelerated using an electric field, a low applied voltage will create a small electric field which lands them on the substrate with little or no thermal effects. Higher applied voltages, however, will result in greater field strengths, which if sufficiently great will result in a transformation of kinetic energy into heat in an amount sufficient to melt the nanoparticles slightly together, leaving gaps between the particles. Similarly, in embodiments where magnetic or paramagnetic nanoparticles are accelerated using a magnetic field, a low magnetic field strength will just land the nanoparticles on the surface with little or no thermal effects, whereas higher magnetic field strengths will result in the transformation of kinetic energy into heat sufficient to melt the nanoparticles slightly together, leaving gaps between the particles. Even higher field strengths (e.g., magnetic, electrical, etc.) will solidify the individual particles into a solid material without gaps. In some embodiments, adhesion of the nanoparticles to the underlying structure and/or to one another each other can be tuned (e.g., by the extent of acceleration). Moreover, layers can be formed, which are tough and adherent or soft and friable.

Where porous inorganic layers are formed, the size distribution of the nanoparticles may have a large effect on the pore-size distribution, with larger particles capable of creating larger pores, which pore sizes may be further tailored through the adjustment of field strength. Sustained drug release may be promoted by creating a uniform porosity throughout the nanoporous layer, which will depend upon both the initial size of the particles as well as upon the melting effect that arises from the field strength.

As a specific example, a system for performing nanoparticle deposition along the lines described above is available from Mantis Deposition Ltd., Thame, Oxfordshire, United Kingdom, who market a high-pressure magnetron sputtering source which is able to generate nanoparticles from a sputter target with as few as 30 atoms up to those with diameters exceeding 15 nm. (A system similar to the Mantis system can be obtained from Oxford Applied Research, Witney, Oxon, UK.) This system is operated at about 5×10⁻⁵ mbar, although the precise operating pressure used will vary widely, depending on the specific process and system that is employed, among other factors. The size of the nanoparticles is affected by several parameters, including the nanoparticle material, the distance between the magnetron surface and the exit aperture (e.g., larger distances have been observed to create larger nanoparticles), gas flow (e.g., higher gas flows have been observed to create smaller nanoparticle sizes), and gas type (e.g., helium has been observed to produce smaller particles than argon). For a particular setting, the size distribution can be measured using a linear quadrapole device placed after the exit aperture of the magnetron chamber. The quadrapole device can also be used in-line to select a narrow nanoparticle size range for deposition. Systems like the Mantis Deposition Ltd. system can produce nanoparticles, a large fraction of which of which (approximately 40% to 80%) have a charge of one electron. Consequently, a magnetic field or a secondary electric field can be used to separate particles of similar weight from one another (because lighter particles are deflected to a greater degree in a given field than are the larger particles of the same charge). For example, the above Mantis Deposition Ltd. system is able to produce charged nanoparticle streams with a very narrow mass distribution. Moreover, it is possible to accelerate the negatively charged particles onto a positively biased surface in order to impact the particles on the surface with elevated kinetic energy. A positively biased grid may also be used to accelerate the particles, allowing the particles to pass through holes in the grid and impinge on the surface. By altering the bias voltage from low to high values the deposited film changes from porous loosely bound nanoparticles to a solid film of metal. Due to the fact that the amount of energy needed to melt the individual nanoparticles is relatively low compared to the energy needed to increase the bulk temperature of an underlying structure, this process is effectively performed at or near room temperature. When using a system like the Mantis Deposition Ltd. system, it has been found that the bias voltage (which may vary, for example, from 10 V to 5000 V) and the particle size (which may vary, for example, from 0.7 nm to 25 nm) has a significant effect upon drug release, with higher voltages and smaller particle sizes yielding coatings with reduced drug release.

As previously indicated, in some PVD embodiments, it may be desirable to change the orientation of the structure (upon which the material is to be deposited) relative to the material stream. For example, a tubular medical device such as a stent may be axially rotated (and, optionally, reciprocated longitudinally) while exposing it to the material stream.

### EXAMPLE

A Nitinol drug eluting spiral is made for an application in the superior femoral artery (SFA). Specifically, a 2130 mm long, 0.30 mm nitinol wire (type S), Memory Metalle GmbH, Am Kesselhaus 5, D-79576 Weil am Rhein, Germany, is shape set into a spiral shape (diameter 4.5 mm, pitch 2 mm at a temperature of 475° C. over the course of 5 minutes).

An electrospun fiber network of polymer nano-fibers is formed on the Nitinol surface, after which the fiber network is covered by an everolimus coating, and a final coating layer of TiOₓ (titania) particles, leaving a porous titiania membrane around the everolimus-coated PEI fibers. The internal PEI fiber network serves both as surface enlarger as well as scaffolding to hold the titania layer intact.

More particularly, polyetherimide (PEI) from Aldrich Co. (St. Louis, Mo.), and Biopol™ polyhydroxybutyrate-valerate (PHBV) from Monsanto Company (St. Louis, Mo.) are mixed in chloroform making respective solutions having 23 wt % PEI and 21 wt % PHBV. These two solutions are mixed to a ratio of 75/25 (PEI/PHBV). The Nitinol spiral is stretched vertically to at or near its full original 2130 mm length using a 500 g weight, and a grounded electrical contact is connected to each end of the stretched wire. A nozzle with a syringe is placed at a distance of 15 cm from the Nitinol wire and connected to a syringe pump (type SP101i, World Precision Instruments, Liegnitzer Str.15, D-10999 Berlin, Germany) and a high voltage supply (Type CS2091, High Voltage Power Solutions, Inc., Dallas, Tex.). The Nitinol wire is rotated at 5 Hz during the spraying process and moved along the axis in a cyclic movement of 12 Hz with an amplitude of 2 mm up and 2.5 mm down. The spraying is carried out at the following settings: 15 kV, 0.05 ml/min, 6 minutes for one cycle. The wire sprayed in this way is thermally treated for 90 minutes at 210° C. in a nitrogen environment to decompose the PHBV component and leave behind a fiber meshwork made of porous PEI fibers on the Nitinol wire. The weight is removed from the wire during the drying process to allow the wire to return to its spiral shape.

In the following step, this fiber PEI network is covered with an Everolimus coating by dissolving Everolimus 2% by weight in a 50:50 mixture of cyclohexanone and acetone. This solution is sprayed onto the porous PEI fibers covering the Nitinol spiral. During the spraying process at a rate of 0.05 mL/min (same syringe pump as above), the Nitinol wire is rotated at 5 Hz and moved up at a speed of 50 cm/minute in order to obtain an everolimus dose of about 100 ug/cm² of covered vessel wall after implantation.

In order to cover the entire assembly with a layer of TiOₓ nanoparticles (which may also include heparin), an aqueous solution containing 0.01 mol/L of titanium tetrachloride and 0.1 mol/L of hydrochloric acid is prepared. Titanium (IV) chloride is added under vigorous stirring to the aqueous solution. The aqueous solution is poured into a microwave reactor (Biotage Advancer, Biotage, Uppsala, Sweden), a 0.4-MPa argon pressure is introduced into the system, and then the reactor is exposed to microwaves for 30 s at 500 Watt power level. The pressure level is maintained at a max of 1.5 bar. An aqueous heparin solution (200 mg/10 ml water) is prepared and added under vigorous stirring to the resulting TiOₓ solution in a 1:1 ratio immediately after the TiOₓ solution is cooled to room temperature. The Nitinol-supported spiral is dip-coated 4 times in the heparin\TiOₓ solution and dried in between dip-coating steps at 70° C. for 1 hour.

Although various embodiments are specifically illustrated and described herein, it will be appreciated that modifications and variations of the present invention are covered by the above teachings and are within the purview of the appended claims without departing from the spirit and intended scope of the invention.

1. An implantable or insertable medical device comprising a substrate, a therapeutic agent disposed over or in said substrate, and a inorganic layer disposed over the therapeutic agent and the substrate, wherein said inorganic layer is a rough inorganic layer, and wherein said inorganic layer is either a porous inorganic layer or is a non-porous inorganic layer that becomes a porous inorganic layer after implantation or insertion of the device into a subject for a sufficient time.

2. The medical device of claim 1, wherein the medical device is selected from a stent, an electrical stimulation lead, a heart valve, a bone scaffold, a soft tissue scaffold, and a balloon assembly.

3. The medical device of claim 1, wherein the substrate is selected from a biodisintegrable metallic substrate and a biostable metallic substrate.

4. The medical device of claim 1, wherein the inorganic layer is selected from a biostable inorganic layer, a biodisintegrable inorganic layer, and an inorganic layer that is partially biodisintegrable and partially biostable.

5 The medical device of claim 1, wherein the inorganic layer is a vapor deposited layer.

6. The medical device of claim 1, wherein the inorganic layer is a metallic layer.

7. The medical device of claim 1, wherein said rough inorganic layer displays the contours of an underlying rough material region.

8. The medical device of claim 7, wherein the underlying rough material region is a rough substrate.

9. The medical device of claim 8, wherein said therapeutic agent is provided in the form of a substantially pure layer between the underlying rough substrate and the overlying inorganic layer.

10. The medical device of claim 7, wherein the underlying rough material region is a rough layer of material that is disposed under the inorganic layer and over the substrate.

11. The medical device of claim 10, wherein said rough layer of material comprises said therapeutic agent.

12. The medical device of claim 10, wherein said rough layer of material comprises interconnected polymeric particles.

13. The medical device of claim 12, wherein said rough layer of material is an electrospray deposited layer.

14. The medical device of claim 10, wherein said rough layer of material comprises a sol-gel derived metal oxide, a sol-gel derived silicon oxide, or combination thereof.

15. The medical device of claim 7, comprising an intervening layer of material between said rough material region and said inorganic layer.

16. The medical device of claim 15, wherein said intervening layer comprises said therapeutic agent.

17. The medical device of claim 15, wherein said intervening layer is disposed between said therapeutic agent and said inorganic layer.

18. The medical device of claim 17, wherein said intervening layer is a biodisintegrable layer.

19. An implantable or insertable medical device comprising a substrate, a therapeutic agent disposed over or in said substrate, and an inorganic layer disposed over the therapeutic agent, wherein said inorganic layer comprises a biostable inorganic phase and a biodisintegrable inorganic phase, and wherein the inorganic layer becomes porous upon implantation or insertion of the device into a subject.

20. The medical device of claim 19, wherein the medical device is selected from a stent and an electrical stimulation lead.

21. The medical device of claim 19, wherein the inorganic layer comprises a biodisintegrable metallic phase and a biostable metallic phase.

22. The medical device of claim 19, wherein the inorganic layer is a vapor deposited layer.

23. The medical device of claim 19, wherein the therapeutic agent is disposed within depressions in said substrate.

24. The medical device of claim 19, wherein the therapeutic agent is in substantially pure form.

25. The medical device of claim 19, wherein the therapeutic agent is provided in a composition that comprises the therapeutic agent and a polymer.
